(19) United States
(12) Patent Application Publication
(10) Pub. No.: US 2014/0316324 A1
(43) Pub. Date: Oct. 23, 2014

(54) Fluid Absorbent Adhesive Articles

(71) Applicant: Avery Dennison Corporation, Glendale, CA (US)

(72) Inventors: Anne Marie Wibaux, Cleveland Heights, OH (US); Vicky Van De Pol, Turnhout (BE); Honorina Repriels, Turnhout (BE)

(73) Assignee: Avery Dennison Corporation, Glendale, CA (US)

(21) Appl. No.: 14/357,307

(22) PCT Filed: Nov. 9, 2012
(86) PCT No.: PCT/US2012/064291

§ 371 Date: May 9, 2014

Related U.S. Application Data
(60) Provisional application No. 61/557,963, filed on Nov. 10, 2011.
(60) Provisional application No. 61/587,244, filed on Jan. 17, 2012.

(51) Int. Cl. A61L 15/58; A61F 13/02

(52) U.S. Cl. CPC A61L 15/585; A61F 13/0283; A61F 13/0253
USPC 602/56; 604/319; 427/2.31

(57) ABSTRACT

A fluid absorbing pressure sensitive adhesive composition and adhesive articles including the fluid absorbing adhesive that have particular utility in the medical field, and in particular, for use with wound dressings is provided. The adhesive composition has superior fluid handling capacity, moisture vapor transmission and skin adhesion. Also described are dressings, cover drapes, and/or sealing components for negative pressure wound therapy applications. The articles include a thin film having a layer of a breathable hydrocolloid adhesive. When used in negative pressure wound therapy applications, the articles are breathable and fluid absorbing.

### CROSS REFERENCED TO RELATED APPLICATIONS

This application claims the benefit of U.S. Provisional Patent Application No. 61/557,963 filed Nov. 10, 2011 and U.S. Provisional Patent Application No. 61/587,244 filed Jan. 17, 2012, which are incorporated herein by reference in their entireties.

### BACKGROUND

The present subject matter relates to a fluid absorbing pressure sensitive adhesive composition and adhesive articles including the fluid absorbing adhesive that have particular utility in the medical field, and in particular, for use with wound dressings. The adhesive composition has superior fluid handling capacity, moisture vapor transmission and skin adhesion.

The present subject matter also relates to a fluid absorbing pressure sensitive adhesive hydrocolloid composition and adhesive articles including the fluid absorbing adhesive that have particular utility in the medical field, and in particular, for use with wound dressings and cover drapes in negative pressure wound therapies (NPWT). The adhesive hydrocolloid composition has superior fluid handling capacity, moisture vapor transmission and skin adhesion properties.

Hydrocolloid containing dressings and adhesive articles are widely used in the treatment of wounds. It is desirable that the hydrocolloid containing article be highly absorbent, have a high moisture vapor transmission rate (MVTR) and at the same time not be too thick, so that a high degree of flexibility and comfort is maintained for the patient.

A wide array of dressings, drapes, and sealing components are used in negative pressure wound therapy (NPWT). In such treatment therapies, typically, dressings, drapes, and sealing components are placed over a desired area of a patient's body, for example a wound area, to form a sealed area for subjecting to reduced pressure. Dressings and drapes may be provided with an adhesive coating along their underside for adhering and sealing the dressing or drape to the patient's skin. Other sealing components may include a transfer tape using medical grade adhesive. In many instances, numerous “leaks” occur between the covered wound area and the external atmosphere. Leaking can occur due to many factors such as non-uniformity of the patient's skin, wrinkles or folds occurring in the dressing or drape, and displacement between the interfacing surfaces, e.g. skin and dressing, as a result of movement or loss in adhesion. As will be appreciated, leaking is undesirable as maintenance of a reduced pressure environment about the wound area can be compromised.

Typically, although a concern, leaking in negative pressure wound therapies is countered by the use of sufficiently sized vacuum pumps. Such pumps have a pumping capacity which exceeds and/or readily accommodates any leaking which may occur along the interface between dressing/drape/component and skin, or other regions.

Furthermore, another strategy developed in this field is the use of relatively thick coatings of adhesive along the underside of dressings and sealing components. As will be appreciated, relatively thick coatings tend to conform to a non-uniform skin surface or accommodate dimensional changes along an interface, and thus promote sealing between a dressing/drape/component and skin.

However, the use of relatively thick coatings of adhesive increases the cost of dressings, drapes, and sealing components. Furthermore, depending upon the coverage of the coating, other properties of the dressing, drape, or component may be detrimentally affected such as breathability, and transmission rates of water vapor and/or oxygen.

In addition, mobile or portable negative pressure wound therapy systems have been developed. Such systems which include a vacuum pump are typically smaller and lighter. As a consequence of improved portability, often the vacuum pump is smaller and has less capacity to accommodate leaks.

Accordingly, a need exists for low cost dressings, drapes, and/or sealing components that are less susceptible to leaks and thus promote formation and maintenance of reduced pressure regions in negative pressure wound therapies.

### SUMMARY

In one embodiment of the present subject matter, there is provided an adhesive composite having superior skin adhesion, breathability and fluid handling capacity. The composite comprises: (i) a polymeric backing layer; and (ii) a fluid absorbing adhesive layer comprising (a) 20-80% by weight of a solvent-based acrylic pressure sensitive adhesive and (b) 20-80% by weight of at least one gelling agent, wherein the thickness of the adhesive layer is about 40 μm to about 300 μm, and the overall adhesive composite has a moisture vapor transmission rate of at least 2000 g/m²/24 hours.

The fluid absorbing adhesive layer may be made up of a single adhesive film or multiple adhesive films laminated together. The multiple adhesive films may have the same composition or different compositions.

In another embodiment of the subject matter, there is provided a multilayer adhesive composite that includes two absorbing layers, the composite having a higher fluid handling capacity than a single adhesive layer composite without significantly increasing the thickness of the composite. The multilayer composite comprises: (i) a polymeric backing layer having a first surface and a second surface; (ii) a first fluid absorbing adhesive layer having a first surface and a second surface comprising a solvent-based acrylic pressure sensitive adhesive, and 20-80% by weight of at least one gelling agent having an average particle size of less than 70 μm, wherein the thickness of the adhesive layer is about 40 μm to about 300 μm and the first surface of the first adhesive layer is adhered to the second surface of the backing layer; and (iii) a second fluid absorbing adhesive layer having a first surface and a second skin-contacting surface comprising a rubber-based pressure sensitive adhesive, wherein the thickness of the adhesive layer is at least 200 μm and the first surface of the second adhesive layer is adhered to the second surface of the first adhesive layer, and wherein the overall adhesive composite has a fluid handling capacity of at least 4000 g/m²/24 hours.

In another aspect, a multilayer medical article adapted for use in negative pressure wound therapy application is provided. The article comprises a polymeric film having a moisture vapor transmission rate (MVTR) of from 1,500 to 14,600 g/m²/24 hours at 38° C. The article also comprises a layer of an adhesive composition disposed on the polymeric film. The adhesive composition includes (i) at least one adhesive component, and (ii) at least one of a moisture absorbing agent and a hydrocolloid. The thickness of the adhesive layer is from 50 to 250 μm.

In another aspect, a method of producing a multilayer medical article adapted for use in negative pressure wound therapy applications is provided. The method comprises providing a polymeric film having a moisture vapor transmission rate (MVTR) of from 1,500 to 14,600 g/m²/24 hours at 38° C. The method also comprises providing an adhesive composition including (i) at least one adhesive component and (ii) at least one of a moisture absorbing agent and a hydrocolloid. The method additionally comprises forming a layer of the adhesive composition on the film such that the layer has a thickness of from 50 to 250 μm.

In yet another aspect, a method of forming a sealed region along a biological surface is provided. The method comprises providing a multilayer medical article including a polymeric film having a moisture vapor transmission rate (MVTR) of from 1,500 to 14,600 g/m²/24 hours at 38° C., a layer of an adhesive composition disposed on the polymeric film, the adhesive composition disposed on the polymeric film, the adhesive composition including (i) at least one adhesive component, and (ii) at least one of a moisture absorbing agent and a hydrocolloid. The thickness of the adhesive layer is from 50 to 250 μm. The method also comprises contacting the adhesive layer to the biological surface to thereby form a sealed region between the article and the biological surface.

As will be realized, the subject matter described herein is capable of other and different embodiments and its several details are capable of modifications in various respects, all without departing from the claimed subject matter. Accordingly, the drawings and description are to be regarded as illustrative and not restrictive.

### BRIEF DESCRIPTION OF THE DRAWINGS

FIG. 1 is a schematic cross-sectional view of an adhesive article in accordance with one embodiment of the subject matter.

FIG. 2 is a schematic cross-sectional view of one embodiment of the adhesive article having two adjacent adhesive layers, each of the adhesive layers comprising a fluid absorbing solvent-based acrylic adhesive.

FIG. 3 is a schematic cross-sectional view of one embodiment of the adhesive article having three adjacent adhesive layers.

FIGS. 4 and 5 are schematic cross-sectional views of embodiments of the adhesive article having two adhesive layers, wherein the adhesive layers have different surface areas.

FIG. 6 is a schematic cross-sectional view of one embodiment of the adhesive article having two adhesive layers, one layer comprising a thin fluid absorbing solvent-based adhesive layer and the other comprising a thicker fluid absorbing rubber-based adhesive layer.

FIG. 7 is a schematic cross-sectional view of an embodiment of the adhesive article having two adhesive layers, one layer comprising a thin fluid absorbing solvent-based adhesive layer and the other comprising a thicker fluid absorbing rubber-based adhesive layer, wherein the adhesive layers have different surface areas.

FIG. 8 is a schematic exploded view of a preferred embodiment article illustrating a thin film substrate layer, an adhesive layer, and a liner covering the adhesive layer.

FIG. 9 is a schematic view of another preferred embodiment article illustrating a thin film substrate layer and a patterned adhesive layer.

### DETAILED DESCRIPTION OF THE EMBODIMENTS

The present subject matter is directed to a fluid absorbing pressure sensitive adhesive comprising a solvent-based acrylic adhesive and at least one gelling agent having an average particle size of less than about 100 μm.

The fluid absorbing adhesive may be coated onto a breathable polymeric backing to provide an adhesive composite having superior fluid handling capacity. In one embodiment, the composite comprises (i) a polymeric backing layer; (ii) a fluid absorbing adhesive layer comprising (a) 20-80% by weight of a solvent-based acrylic pressure sensitive adhesive and (b) 20-80% by weight of at least one gelling agent which is preferably a super absorbent polymer or hydrocolloid having an average particle size of less than 150 μm, wherein the thickness of the adhesive layer is about 40 μm to about 300 μm, and the overall adhesive composite has a fluid handling capacity of at least about 2000 g/m²/24 hours and a moisture vapor transmission rate of at least 1100 g/m²/24 hours. In one embodiment, the moisture vapor transmission rate is at least 1200 g/m²/24 hours. In other embodiments, the moisture vapor transmission rate is at least 2000 g/m²/24 hours.

In another embodiment, the composite comprises (i) a polymeric backing layer; (ii) a fluid absorbing adhesive layer comprising (a) 20-80% by weight of a solvent-based acrylic pressure sensitive adhesive and (b) 20-80% by weight of a gelling agent which preferably is at least one super absorbent polymer or hydrocolloid having an average particle size of less than 70 μm, wherein the thickness of the adhesive layer is about 40 μm to about 300 μm, and the overall adhesive composite has a moisture vapor transmission rate of at least 2000 g/m²/24 hours, without using pattern coating. The static absorption of the adhesive is greater than 600 g/m²/24 hours.

In still other embodiments, the composite comprises (i) a polymeric backing layer; (ii) a fluid absorbing adhesive layer comprising (a) 20-80% by weight of a solvent-based acrylic pressure sensitive adhesive and (b) 20-80% by weight of at least one gelling agent which is preferably a super absorbent polymer or hydrocolloid having an average particle size of less than 150 μm, wherein the thickness of the adhesive layer is about 40 μm to about 300 μm, and preferably from about 80 μm to about 100 μm and the overall adhesive composite has a moisture vapor transmission rate of at least 2000 g/m²/24 hours.

Although not wishing to be bound to any particular theory, it is believed that the adhesive composites exhibit a MVTR of at least 2000 g/m²/24 hours due to the presence of the gelling agent in the adhesive. That is, any moisture or water in the adhesive has an improved transit through the thickness of the adhesive layer because the gelling agent increases the uptake of moisture into the adhesive and therefore increases the MVTR. Control or design of the particular MVTR can be limited or further controlled by selection of the film laminated on top of the adhesive layer.

As used herein, the term “Fluid Handling Capacity” means the combined ability of the article to take up moisture and to evaporate it to the environment. The fluid handling capacity of the composite in one embodiment is at least about 2500 g/m²/24 hours, or at least about 3500 g/m²/24 hours at an adhesive layer thickness of about 80 μm to about 300 μm.

The static absorption of the composite, in one embodiment, is greater than 600 g/m²/24 hours, or at least about 700 g/m²/24 hours, or at least about 850 g/m²/24 hours, or at least about 1000 g/m²/24 hours.

### Acrylic Adhesive

The solvent-based acrylic adhesive may be any pressure sensitive adhesive that is capable of adhering to mammalian skin and that is free of ingredients known to cause undue irritation or toxicity to mammals.

Useful acrylate copolymers may or may not be self-crosslinking and are formed from at least two monomers chosen from: (1) hydroxyalkyl esters of acrylic or methacrylic acid in which the alkyl group comprises 2 to 4 carbon atoms, such as 2-hydroxyethyl acrylate, 2-hydroxyethyl methacrylate, 2-hydroxypropyl acrylate and 2-hydroxypropyl methacrylate; (2) alkyl esters of acrylic or methacrylic acid in which the alkyl group of the ester comprises 4 to 18 carbon atoms, such as n-butyl acrylate or methacrylate, isopropyl acrylate or methacrylate, n-hexyl methacrylate and 2-ethylhexyl acrylate; (3) α,β-unsaturated monocarboxylic or dicarboxylic acids, their anhydrides and their alkyl or alkenyl esters in which the alkyl group contains from 1 to 3 carbon atoms and the alkenyl group contains from 2 to 5 carbon atoms, such as acrylic acid, itaconic acid, maleic acid, maleic anhydride, alkyl methacrylate and the diethyl esters of fumaric or maleic acid; (4) vinyl monomers, such as vinyl acetate, acrylonitrile, vinyl propionate, vinylpyrrolidone and styrene; (5) monomers containing a functional group selected from amido, amino and epoxy groups, for example, acrylamide, N-butylacrylamide, alkylaminoalkyl and aminoalkyl derivatives of acrylic or methacrylic acid, such as amino-ethyl acrylate, aminoethyl methacrylate and 2-(dimethylamino) ethyl methacrylate, glycidyl methacrylate and glycidyl acrylate; (6) alkoxyalkyl esters of acrylic or methacrylic acid, for example methoxyethyl acrylates or methacrylates, butoxyethyl acrylates or methacrylates, methoxypropylene glycol acrylates or methacrylates and methoxypolyethylene glycol acrylates or methacrylates; and (7) hexamethylene glycol dimethacrylate.

As these copolymers can be self-crosslinking, they may also contain a crosslinking agent selected from those generally used by those skilled in the art, for example, organic peroxides, polyisocyanates, chelates or metals such as titanium or aluminum, or metal acetylacetonates, such as those of zinc, magnesium and aluminum.

These adhesive acrylate copolymers may take the form of solutions in a solvent system consisting of a single organic solvent or a mixture of several solvents, which contain about 25% to about 55% by weight copolymers. Examples of suitable solvents include aromatic solvents such as toluene, xylene, etc. Suitable aliphatic solvents include esters such as ethyl acetate, propyl acetate, isopropyl acetate, butyl acetate, etc.; ketones such as methyl ethyl ketone, acetone, etc.; aliphatic hydrocarbons such as heptanes, hexane, pentane, etc.

There can be included in the adhesive composition additive materials that do not affect the basic properties of the adhesive. Fillers, tackifiers, antioxidants, stabilizers, and the like may be added to the formulate adhesive. Further, pharmaceutically active components, such as for example, antimicrobials, anti-inflammatory agents, analgesic agents, anesthetics, or other pharmaceutically acceptable compounds, which do not affect the basic properties of the adhesive can be included in the adhesive layer in a pharmaceutically effective amount.

An example of a useful commercially available adhesive is DURO-TAK 380-2819 from National Starch, which is a self-crosslinking solution acrylic pressure sensitive adhesive containing 40% by weight solids in a solvent blend of ethyl acetate/isopropanol/heptanes/toluene/pentanedione.

### Super Absorbent Polymer

As noted, the gelling agent may in certain embodiments be a super absorbent polymer.

The super absorbent polymer (SAP) useful in the adhesive composition comprises a water-swellable, hydrogel-forming absorbent polymer capable of absorbing large quantities of liquids such as water, body fluids (e.g., urine, blood), and the like. Additionally, the SAP is capable of retaining such absorbed fluids under moderate pressures. Typically the SAP absorbs many times its own weight in water, preferably at least 50 times, more preferably at least 100 times, most preferably at least 150 times its weight in water. Additionally, the SAP exhibits good saline fluid absorption under load and high saline fluid absorption capacity. Typically the SAP absorbs at least 10 times, preferably at least 30 times, more preferably at least 50 times its weight in saline fluid. Even though the SAP is capable of absorbing many times its own weight in water and/or saline, it does not dissolve in these fluids.

The ability of the SAP to absorb water and/or saline fluid is related to the degree of crosslinking present in the SAP. Increasing the degree of crosslinking increases the SAP's total fluid holding capacity under load. The degree of crosslinking is preferably optimized to obtain a composition in which the rate and amount of absorbency are optimized. Preferred SAPs are at least 10%, more preferably from about 10% to about 50%, most preferably from about 20% to 40% crosslinked. Examples of suitable SAPs include crosslinked and polymerized α,β-beta ethylenically unsaturated mono- and dicarboxylic acids and acid anhydride monomers including, e.g., acrylic acid, methacrylic acid, crotonic acid, maleic acid/anhydride, itaconic acid, fumaric acid, and combinations thereof.

Super absorbent polymers useful in the subject matter include, e.g., crosslinked acrylate polymers, crosslinked products of vinyl alcohol-acrylate copolymers, crosslinked products of polyvinyl alcohols grafted with maleic anhydride, cross-linked products of acrylate-methacrylate copolymers, crosslinked saponification products of methyl acrylate-vinyl acetate copolymers, crosslinked products of starch acrylate graft copolymers, crosslinked saponification products of starch acrylonitrile graft copolymers, crosslinked products of carboxymethyl cellulose polymers and crosslinked products of isobutylene-maleic anhydride copolymers, and combinations thereof.

The super absorbent particles preferably are spherical and have an average particle size of from about 1 micrometer (μm) to about 400 (μm). Preferably the particles have an average particle size of from about 20 μm to about 200 μm, and more preferably from 20 μm to 150 μm. In one embodiment, the particle size of the particles is less than 150 μm, or less than 100 μm. Useful commercially available super absorbent particles include, e.g., sodium polyacrylate super absorbent particles available under the AQUA KEEP series of trade designations including, e.g., particles having an average particle size of from about 20 μm to about 30 μm available under the trade designation AQUA KEEP 1 OSH-NF, particles having an average particle size of from 200 μm to 300 μm available under the trade designation AQUA KEEP 10SH-P, particles having an average particle size of from 320 μm to 370 μm available under the trade designation AQUA KEEP SA60S, particles having an average particle size of from 350 μm to 390 μm available under the trade designations AQUA KEEP SA60SX, SA55SX π and SA 60SL II, and particles having an average particle size of from 250 μm to 350 μm available under the trade designation AQUA KEEP SA60N TYPE II from Sumitomo Seika Chemicals Col, Ltd. (Japan). Also available super absorbent materials are Luquasorb 1010 and Luquasorb 1030 from BASF, Ludwigshafen, Germany.

In one embodiment, the adhesive contains about 20% by weight to about 80% by weight of a super absorbing polymer. In another embodiment, the adhesive contains about 40 to about 60% by weight of a super absorbing polymer.

### Hydrocolloids

As noted, the gelling agent may in certain embodiments be a hydrocolloid, and thus the adhesive composition may include a hydrocolloid. The hydrocolloids enable the final composition to adhere to moist body surfaces. This phenomenon is termed “wet tack”. One or more water swellable hydrocolloids may also be present. The hydrocolloid may be linear or crosslinked. Suitable hydrocolloids include synthetic hydrocolloids such as sodium carboxymethyl cellulose, and natural products such as gelatin, pectin, guar gum, locust bean gum, tragacanth gum, gum karaya, starches, gum arabic, alginic acid and its sodium and/or calcium salts. Other synthetic hydrocolloids such as polyvinyl alcohol, polyvinyl acetate, polyvinyl pyrollidone, polyacrylic acid, polyhydroxyalkyl acrylates, polyacrylamides, high molecular weight polyethylene glycols and polypropylene glycols are useful. Others hydrocolloids include crosslinked or crystalline sodium carboxymethyl cellulose, crosslinked dextran and starch-acrylonitrile graft copolymer.

The hydrocolloid particles preferably have an average particle size of from about 1 micrometer (μm) to about 400 (μm). Preferably the particles have an average particle size of from about 20 μm to about 200 μm, and more preferably from 20 μm to 150 μm. In one embodiment, the particle size of the particles is less than 150 μm, or less than 100 μm.

### Backing

The backing layer is made of a thin polymeric elastic or flexible film that is water vapor permeable. The film may be liquid and/or bacteria impermeable. The backing layer may comprise polyurethane, elastomeric polyester, blends of polyurethane and polyester, polyvinyl chloride, polyether-amide block copolymer and porous polyethylene. In one embodiment, the backing is a polyurethane film.

Suitable backing layers are thin and have good conformability. In one embodiment, the thickness of the backing is in the range of about 10 μm to about 75 μm, or about 15 μm to about 45 μm, or about 20 μm to about 30 μm. The moisture vapor transmission rate (MVTR) of the backing layer alone is within the range of about 1500 to about 14600 g/m²/24 hours, or from about 2500 to about 10000 g/m²/24 hours at 38° C.

### Release Liner

In one embodiment, the adhesive article includes a release-coated liner on the skin-contacting side, which is retained in place prior to use and is removed just prior to application to the user's skin. The release-coated liner may be any release-coated liner known in the art that is compatible with the pressure sensitive adhesive of the skin-contacting side of the adhesive article.

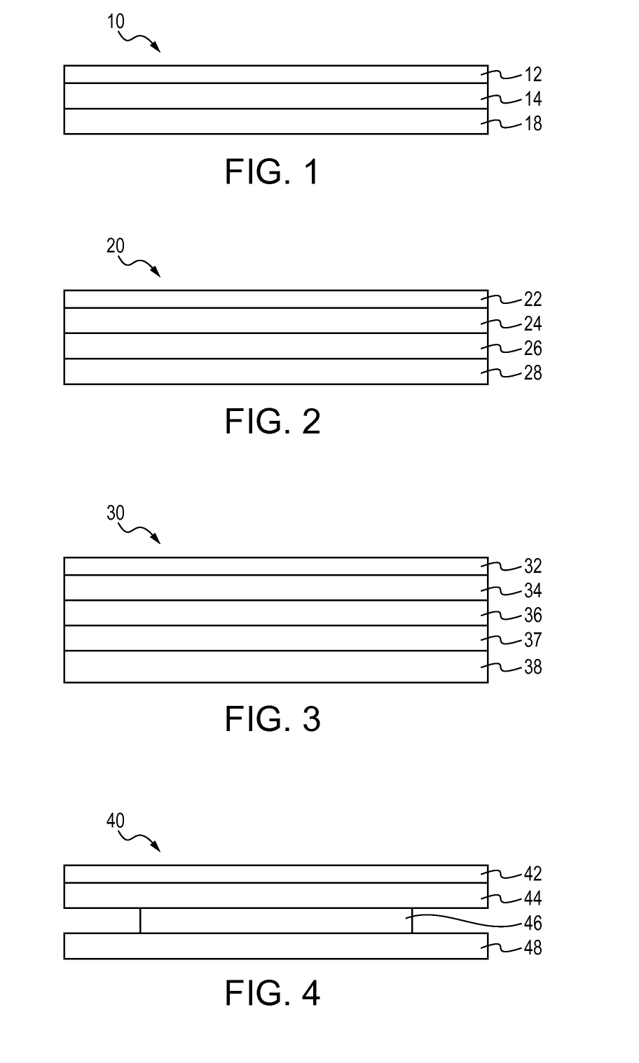

FIG. 1 is a schematic cross-sectional view of a skin-contacting adhesive article 10 in accordance with one embodiment of the subject matter. The adhesive article 10 includes a breathable backing layer 12, adhesive layer 14 adhered to the backing layer, and an optional release liner 18 removably adhered to the adhesive layer. Prior to use, the release liner 18, if present, is removed to allow adhesive layer 14 to be applied to the skin of the user. Adhesive layer 14 contains a solvent-based acrylic adhesive having at least one super absorbing polymer dispersed therein. The thickness of adhesive layer 14 can be in the range of about 40 to about 200 μm. In one embodiment, the thickness of the adhesive layer less than about 200 μm, or within the range of about 60 to about 150 μm. With the solvent-based adhesives, coat weights greater than about 200 μm may result in undesirably high levels of residual solvent after drying of the coated adhesive.

FIG. 2 is a schematic cross-sectional view of a skin-contacting adhesive article 20 in accordance with another embodiment of the subject matter. The adhesive article 20 includes a breathable backing layer 22, a first adhesive layer 24 adhered to the backing layer, a second adhesive layer 26 adhered to the first adhesive layer and an optional release liner 28 removably adhered to the second adhesive layer 26. Prior to use, the release liner 28, if present, is removed to allow adhesive layer 26 to be applied to the skin of the user. Each of adhesive layers 24 and 26 contains a solvent-based acrylic adhesive having at least one super absorbing polymer dispersed therein. The super absorbing polymer content of adhesive layer 24 may be in the range of about 20% to about 80% by weight and the super absorbing polymer content of adhesive layer 26 may be in the range of about 20% to about 60% by weight, based on the total weight of the adhesive layer. The thickness of each adhesive layer may be in the range of about 40 to about 200 μm, so that the overall thickness of the adhesive is within the range of about 80 to about 400 μm, or within the range of about 150 to about 300 μm.

FIG. 3 is a schematic cross-sectional view of a skin-contacting adhesive article 30 in accordance with a further embodiment of the subject matter. The adhesive article 30 includes a breathable backing layer 32, a first adhesive layer 34 adhered to the backing layer, a second adhesive layer 36 adhered to the first adhesive layer and a third adhesive layer 37 adhered to the second adhesive layer. The adhesive article may include a release liner 38 removably adhered to the third adhesive layer. Each of adhesive layers 34, 36 and 37 contains a solvent-based acrylic adhesive having at least one super absorbing polymer dispersed therein. The super absorbing polymer content of adhesive layers 34 and 36 may be in the range of about 20% to about 80% by weight and the super absorbing polymer content of adhesive layer 37 may be in the range of about 20% to about 60% by weight, based on the total weight of the adhesive layer. The thickness of each adhesive layer may be in the range of about 40 to about 200 μm. The multiple layers of adhesive provide higher performance with respect to fluid handling capacity, moisture vapor transmission and static absorption.

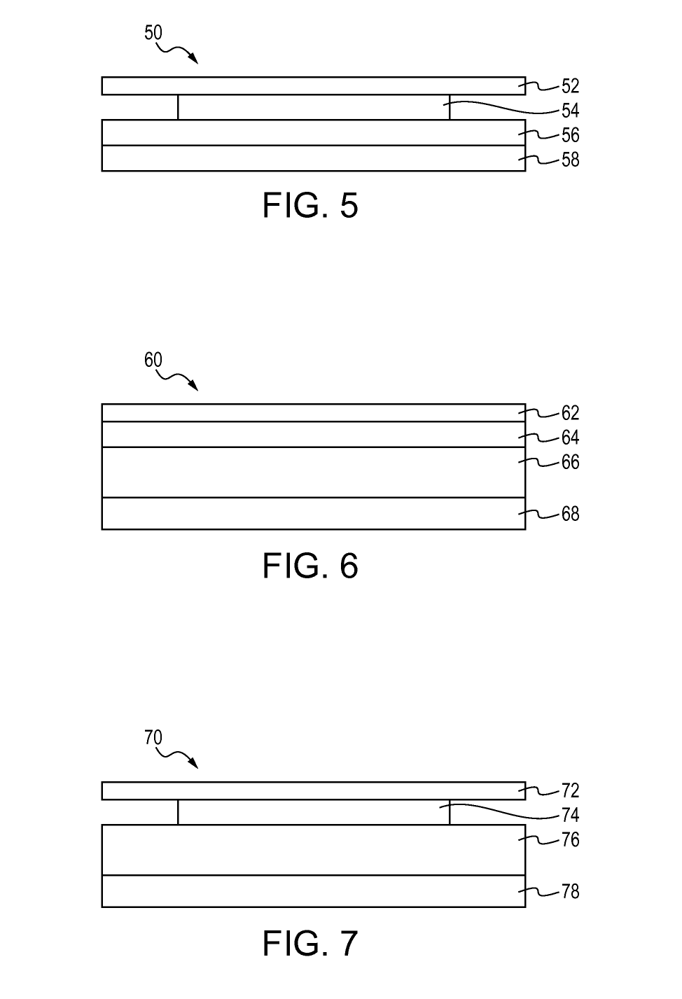

FIGS. 4 and 5 illustrate embodiments similar to that shown in FIG. 2, with the exception that the surface area of the two adhesive layers may be different. Specifically, the adhesive article 40 of FIG. 4 includes a breathable backing layer 42, a first adhesive layer 44 adhered to the backing layer, a second adhesive layer 46 adhered to the first adhesive layer and an optional release liner 48 removably adhered to the second adhesive layer 46. Each of adhesive layers 44 and 46 contains a solvent-based acrylic adhesive having at least one super absorbing polymer dispersed therein. The surface area of adhesive layer 46 is less than the surface area of adhesive layer 44. In another embodiment shown in FIG. 5, the adhesive article 50 includes a breathable backing layer 52, a first adhesive layer 54 adhered to the backing layer, a second adhesive layer 56 adhered to the first adhesive layer and an optional release liner 58 removably adhered to the second adhesive layer 56. The surface area of adhesive layer 54 is less than the surface area of adhesive layer 56.

The fluid absorbing solvent-based acrylic adhesive may be combined with another absorbing adhesive layer to form an adhesive article having improved absorbing properties without significantly increasing the overall thickness of the adhesive article. For example, a thin layer of the fluid absorbent solvent-based acrylic adhesive may be combined with a relatively thicker layer of another fluid absorbing adhesive layer, such as a rubber-based, hydrocolloid containing adhesive layer to increase the fluid handling capacity of the rubber-based adhesive.

Examples of rubber-based adhesives may include those comprising solid rubbers such as linear or radial A-B-A block copolymers or mixtures of these A-B-A block copolymers with simple A-B block copolymers. However, the proportion of A-B block copolymers, relative to the A-B-A block copolymers, should not normally exceed 85% by weight of the (total) block copolymers. In one embodiment, the proportion is in the range from about 35 to about 85% by weight of the block copolymers, and in another embodiment, the proportion is from about 55 to about 75% by weight of the block copolymers. In one embodiment, lower amounts such as 10 to 35% by weight of the block copolymers are used. These block copolymers can be based on styrene-butadiene, styrene-isoprene, and hydrogenated styrene-diene copolymers such as styrene ethylene-butylene. Suitable styrene-diene copolymers are exemplified by a blend of linear styrene-isoprene-styrene triblock copolymer and linear styrene-isoprene diblock copolymer. Such a material is available from Kraton Polymers as KRATON® D-1161K and has a bound styrene content of about 15% and a diblock content of 17%. A second example is a blend of linear styrene-isoprene-styrene triblock copolymer and linear styrene-isoprene diblock copolymer available from Shell Chemical as KRATON® D-1117 and which has a bound styrene content of about 17% and a diblock content of 33%.

An example of a suitable hydrogenated styrene-diene copolymer is a thermoplastic elastomer comprising a blend of clear linear triblock and diblock copolymer based on styrene and ethylene-butylene with a bound styrene of 14% mass. Such a material is commercially available from Shell Chemical Company as KRATON® G-1657. Another example is KRATON® G-1652 from Shell Chemical Company, which is a thermoplastic elastomer comprised of a clear linear triblock copolymer based on styrene and ethylene-butylene, S-E/B-S, with a bound styrene content of about 30% by weight. Also suitable are polymers in which there is a combination of chemically saturated blocks and chemically unsaturated blocks. For example, a branched copolymer consisting of two polyisoprene chains attached to the rubber midblock of a styrene/ethylene-butylene/styrene triblock copolymer. Such a material, for example, is available from Shell Chemical Company having a styrene content of 18%, and isoprene content of 36% and an ethylene-butylene content of 46% by weight. Also, a low styrene synthetic copolymer of butadiene and styrene, commonly called SBR rubber, can be used as a solid rubber.

In one embodiment, liquid rubbers may be added to the adhesive material to adjust or control the adhesive or other characteristics. Liquid rubbers useful in this embodiment of the subject matter include synthetic liquid isoprene rubber, depolymerized natural rubber, various functionally terminated synthetic liquid isoprene-styrene rubbers and liquid isoprene rubbers, liquid isoprene-styrene copolymer, liquid isoprene-butadiene copolymer, liquid butadiene-styrene copolymer and hydrogenated versions of these materials such as liquid ethylene-propylene-styrene. These liquid rubbers are generally compatible with the solid rubber. The liquid rubbers typically have a molecular weight of 25,000 to 50,000, a glass transition temperature of less than −50° C., and a viscosity at 38° C. of 50 to 10,000 Pas. A block copolymer of styrene and isoprene having a styrene content of about 13% and an isoprene content of about 87%, a glass transition of about −60° C., a melt viscosity of about 240 Pas at 50° C. and which is commercially available from Shell Chemical Company as LIR310, is particularly useful in the practice of the subject matter. Within the adhesive material, in one embodiment, the weight ratio of solid rubber to liquid rubber is in the range from about 100:1 to about 1:2, and is varied in order to obtain the desired degree of adhesiveness and tackiness. In one embodiment, the weight ratio of solid rubber to liquid rubber is in the range from about 50:1 to about 5:1, and in another embodiment, from about 20:1 to about 10:1.

Optionally, an elastomeric polymer such as butyl rubber or high molecular weight polyisobutylene may also be blended into the adhesive material. The optional butyl rubber may be used in the viscosity average molecular weight range of 200,000 to 600,000 and is exemplified by the grades Butyl 065 or Butyl 077, both available from Exxon Chemical. The optional high molecular weight polyisobutylene may be used in the viscosity average molecular weight range of 800,000 to 2,500,000 and is exemplified by the VISTANEX® MM series of products, available from Exxon Chemical, with the MM L-80 grade being a preferred grade for the optional high molecular weight polyisobutylene. The optional high molecular weight rubbers, blended as is indicated above, may be added in amounts suitable to modify various properties of the final formulation and may be from 0% to about 50% of the total weight of the adhesive material, and in one embodiment from about 0.5% to about 25% of the total weight of the adhesive material, and in one embodiment from about 5% to about 10% of the total weight of the adhesive material. The optional low molecular weight polybutenes and/or mineral oil may be added in amounts from 0% to about 20% of the weight of the adhesive material and in one embodiment from about 0.5% to about 10% of the total weight of the adhesive material, and in one embodiment from about 0.5% to about 5% of the total weight of the adhesive material.

FIG. 6 is a schematic cross-sectional view of a skin-contacting adhesive article 60 having multiple layers of different adhesives in accordance with one embodiment of the subject matter. The adhesive article 60 includes a breathable backing layer 62, a first adhesive layer 64 adhered to the backing layer wherein the first adhesive layer 64 contains a solvent-based acrylic adhesive having at least one super absorbing polymer dispersed therein. The super absorbing polymer content of adhesive layer 64 may be in the range of about 20% to about 80% by weight based on the total weight of the adhesive layer 64. The thickness of the first adhesive layer 64 may be in the range of about 60 to about 200 μm. A second adhesive layer 66 is adhered to the first adhesive layer 64. Adhesive layer 66 may be a thicker layer of another absorbing adhesive, for example, a 200 μm to 1000 μm layer of a rubber-based hydrocolloid adhesive. The adhesive article may contain an optional release liner 68 removably adhered to the second adhesive layer 66. Prior to use, the release liner 68, if present, is removed to allow adhesive layer 66 to be applied to the skin of the user.

FIG. 7 illustrates an embodiment similar to that of FIG. 6, with the exception that the surface area of the first adhesive is less than the surface area of the second adhesive. Specifically, the adhesive article 70 includes a breathable backing layer 72, a first adhesive layer 74 adhered to the backing layer wherein the first adhesive layer 74 contains a solvent-based acrylic adhesive having at least one super absorbing polymer dispersed therein. A second adhesive layer 76 is adhered to the first adhesive layer 74. Adhesive layer 76 may be a thicker layer of another absorbing adhesive. Optional release liner 78 is removably adhered to the second adhesive layer 76. The surface area of first adhesive layer 74 is less than the surface area of second adhesive layer 76.

### Negative Pressure Wound Therapy and Related Articles

In additional aspects, the present subject matter relates to articles adapted for use in negative pressure wound therapies.

The preferred embodiment articles comprise (i) a relatively thin film substrate layer, (ii) a coating of a breathable hydrocolloid adhesive composition on the substrate, and (iii) one or more optional liners on the adhesive composition. The article may be provided in a wide array of shapes, sizes, and configurations depending upon the end use application of the article. In certain embodiments the articles are in the form of dressings or drapes for use in negative pressure wound therapies. Dressings and drapes include a uniform coating of the adhesive and typically non-patterned in at least one region, along an underside or face of the substrate layer. Dressings and drapes are typically cut or otherwise appropriately sized by a medical practitioner prior to application. Dressings and drapes, prior to cutting, are available in a wide range of sizes such as square shapes of 100 mm by 100 mm or larger or rectangular shapes of 100 mm by 200 mm or larger. The term “drape” as used in the field typically refers to even larger articles. The preferred embodiment articles may also be provided in the form of smaller sealing components which do not require cutting or sizing. It will be appreciated that the various embodiments and aspects described herein are not limited to dressings, drapes, or sealing components described herein. Instead, a wide range of articles are contemplated for use in negative pressure wound therapies.

### Substrate

The substrate layer is made of a thin polymeric elastic or flexible film that is water vapor permeable. The substrate can be selected from any of the previously noted backing layers, or one or more of the substrates described herein. The film may be liquid and/or bacteria impermeable. The substrate layer may comprise polyurethane, elastomeric polyester, blends of polyurethane and polyester, polyvinyl chloride, polyether-amide block copolymer, porous polyethylene, and combinations thereof. In one embodiment, the substrate is a polyurethane film.

Suitable substrate layers are thin and have good conformability. In one embodiment, the thickness of the substrate is in the range of about 10 μm to about 75 μm, or about 15 μm to about 45 μm, or about 20 μm to about 30 μm. In certain embodiments, the thickness of the substrate is 25 μm. The moisture vapor transmission rate (MVTR) of the substrate layer alone is within the range of about 1500 to about 14,600 g/m²/24 hours, or from about 2500 to about 10,000 g/m²/24 hours, at 38° C. However, it will be appreciated that the present subject matter includes the use of films exhibiting MVTRs less than 1,500 g/m²/24 hours.

### Breathable Hydrocolloid Adhesive

The breathable hydrocolloid adhesive composition preferably comprises (i) one or more adhesive components, and (ii) one or more moisture absorbing agents, and/or at least one hydrocolloid. As explained in greater detail herein, the adhesive may comprise additional components.

The adhesive component is preferably a solvent-based acrylic adhesive and may be any pressure sensitive adhesive that is capable of adhering to mammalian skin and that is free of ingredients known to cause undue irritation or toxicity to mammals.

Useful acrylate copolymers for use in the preferred acrylic adhesive are formed from at least two monomers chosen from: (1) hydroxyalkyl esters of acrylic or methacrylic acid in which the alkyl group comprises 2 to 4 carbon atoms, such as 2-hydroxyethyl acrylate, 2-hydroxyethyl methacrylate, 2-hydroxypropyl acrylate and 2-hydroxypropyl methacrylate; (2) alkyl esters of acrylic or methacrylic acid in which the alkyl group of the ester comprises 4 to 18 carbon atoms, such as n-butyl acrylate or methacrylate, isopropyl acrylate or methacrylate, n-hexyl methacrylate and 2-ethylhexyl acrylate; (3) α:β-unsaturated monocarboxylic or dicarboxylic acids, their anhydrides and their alkyl or alkenyl esters in which the alkyl group comprises from 1 to 3 carbon atoms and the alkenyl group comprises from 2 to 5 carbon atoms, such as acrylic acid, itaconic acid, maleic acid, maleic anhydride, alkyl methacrylate and the diethyl esters of fumaric or maleic acid; (4) vinyl monomers, such as vinyl acetate, acrylonitrile, vinyl propionate, vinylpyrrolidone and styrene; (5) monomers containing a functional group selected from amido, amino and epoxy groups, for example, acrylamide, N-butylacrylamide, alkylaminoalkyl and aminoalkyl derivatives of acrylic or methacrylic acid, such as amino-ethyl acrylate, aminoethyl methacrylate and 2-(dimethylamino) ethyl methacrylate, glycidyl methacrylate and glycidyl acrylate; (6) alkoxyalkyl esters of acrylic or methacrylic acid, for example methoxyethyl acrylates or methacrylates, butoxyethyl acrylates or methacrylates, methoxypropylene glycol acrylates or methacrylates and methoxypolyethylene glycol acrylates or methacrylates; and (7) hexamethylene glycol dimethacrylate.

As these copolymers can be self-crosslinking, they may also contain a crosslinking agent selected from those generally used by those skilled in the art, for example, organic peroxides, polyisocyanates, chelates or metals such as titanium or aluminum, or metal acetylacetonates, such as those of zinc, magnesium and aluminum.

These adhesive acrylate copolymers may take the form of solutions in a solvent system consisting of a single organic solvent or a mixture of several solvents, which contain from about 25% to about 55% by weight copolymers. Examples of suitable solvents include aromatic solvents such as toluene, xylene, etc. Suitable aliphatic solvents include esters such as ethyl acetate, propyl acetate, isopropyl acetate, butyl acetate, etc.; ketones such as methyl ethyl ketone, acetone, etc.; aliphatic hydrocarbons such as heptanes, hexane, pentane, etc.

In certain embodiments, the adhesives for use in the various medical articles described herein are acrylic or acrylate based adhesives. However, it will be appreciated that in certain embodiments, the adhesive compositions can include other adhesive systems besides or in addition to acrylic systems.

There can be included in the adhesive composition additive materials that do not affect the basic properties of the adhesive. Fillers, tackifiers, antioxidants, stabilizers, and the like may be added to the formulate adhesive. Further, pharmaceutically active components, such as for example, antimicrobials, anti-inflammatory agents, analgesic agents, anesthetics, or other pharmaceutically acceptable compounds, which do not affect the basic properties of the adhesive can be included in the adhesive layer in a pharmaceutically effective amount.

An example of a useful commercially available adhesive is DURO-TAK 380-2819 from National Starch, which is a self-crosslinking solution acrylic pressure sensitive adhesive containing 40% by weight solids in a solvent blend of ethyl acetate/isopropanol/heptanes/toluene/pentanedione.

The moisture absorbing agent may in certain embodiments be a super absorbent polymer, and/or other absorbing agents. The super absorbent polymer can be as previously described, which is generally as follows.

The super absorbent polymer (SAP) useful in the adhesive composition comprises a water-swellable, hydrogel-forming absorbent polymer capable of absorbing large quantities of liquids such as water, body fluids (e.g., urine, blood), and the like. Additionally, the SAP is capable of retaining such absorbed fluids under moderate pressures. Typically the SAP absorbs many times its own weight in water, preferably at least 50 times, more preferably at least 100 times, most preferably at least 150 times its weight in water. Additionally, the SAP exhibits good saline fluid absorption under load and high saline fluid absorption capacity. Typically the SAP absorbs at least 10 times, preferably at least 30 times, more preferably at least 50 times its weight in saline fluid. Even though the SAP is capable of absorbing many times its own weight in water and/or saline, it does not dissolve in these fluids.

The ability of the SAP to absorb water and/or saline fluid is related to the degree of crosslinking present in the SAP. Increasing the degree of crosslinking increases the SAP's total fluid holding capacity under load. The degree of crosslinking is preferably adjusted to obtain a composition in which the rate and amount of absorbency are provided as desired. Preferred SAPs are at least 10%, more preferably from about 10% to about 50%, most preferably from about 20% to 40% crosslinked. These crosslinking percentages are degrees or extents of crosslinking in which 100% crosslinking represents the maximum degree or extent of crosslinking attainable by the particular material under consideration. Examples of suitable SAPs include crosslinked and polymerized α,β-beta ethylenically unsaturated mono- and dicarboxylic acids and acid anhydride monomers including, e.g., acrylic acid, methacrylic acid, crotonic acid, maleic acid/anhydride, itaconic acid, fumaric acid, and combinations thereof.

Super absorbent polymers useful in the preferred embodiment adhesive compositions include, e.g., crosslinked acrylate polymers, crosslinked products of vinyl alcohol-acrylate copolymers, crosslinked products of polyvinyl alcohols grafted with maleic anhydride, cross-linked products of acrylate-methacrylate copolymers, crosslinked saponification products of methyl acrylate-vinyl acetate copolymers, crosslinked products of starch acrylate graft copolymers, crosslinked saponification products of starch acrylonitrile graft copolymers, crosslinked products of carboxymethyl cellulose polymers and crosslinked products of isobutylene-maleic anhydride copolymers, and combinations thereof.

The moisture absorbing component which for example is a super absorbent polymer, is typically in a particulate form. The particles are preferably spherical and have an average particle size of from about 1 μm to about 400 μm. Preferably the particles have an average particle size of from about 20 μm to about 400 μm, preferably from about 20 μm to about 200 μm, and more preferably from 20 μm to 150 μm. In one embodiment, the particle size of the particles is less than 150 μm, or less than 100 μm. Useful commercially available super absorbent particles include, e.g., sodium polyacrylate super absorbent particles available under the AQUA KEEP series of trade designations including, e.g., particles having an average particle size of from about 20 μm to about 30 μm available under the trade designation AQUA KEEP 1 OSH-NF, particles having an average particle size of from 200 μm to 300 μm available under the trade designation AQUA KEEP 10SH-P, particles having an average particle size of from 320 μm to 370 μm available under the trade designation AQUA KEEP SA605, particles having an average particle size of from 350 μm to 390 μm available under the trade designations AQUA KEEP SA60SX, SA55SX π and SA 605L II, and particles having an average particle size of from 250 μm to 350 μm available under the trade designation AQUA KEEP SA60N TYPE II from Sumitomo Seika Chemicals Col, Ltd. (Japan). Also available super absorbent materials are Luquasorb 1010 and Luquasorb 1030 from BASF, Ludwigshafen, Germany.

In one embodiment, the adhesive contains about 20% by weight to about 80% by weight of a super absorbing polymer. In another embodiment, the adhesive contains about 40 to about 60% by weight of a super absorbing polymer.

The hydrocolloids enable the final composition to adhere to moist body surfaces. This phenomenon is termed “wet tack”. One or more water swellable hydrocolloids may also be present. The hydrocolloid may be linear or crosslinked. Suitable hydrocolloids include synthetic hydrocolloids such as sodium carboxymethyl cellulose, and natural products such as gelatin, pectin, guar gum, locust bean gum, tragacanth gum, gum karaya, starches, gum arabic, alginic acid and its sodium and/or calcium salts. Other synthetic hydrocolloids such as polyvinyl alcohol, polyvinyl acetate, polyvinyl pyrollidone, polyacrylic acid, polyhydroxyalkyl acrylates, polyacrylamides, high molecular weight polyethylene glycols and polypropylene glycols are useful. Others hydrocolloids include crosslinked or crystalline sodium carboxymethyl cellulose, crosslinked dextran and starch-acrylonitrile graft copolymer.

The hydrocolloid(s) is typically in particulate form and preferably has an average particle size of from about 1 μm to about 400 μm. Preferably the particles have an average particle size of from about 20 μm to about 200 μm, and more preferably from 20 μm to 150 μm. In one embodiment, the particle size of the particles is less than 150 μm, or less than 100 μm.

The thickness of the adhesive composition disposed on the substrate is preferably from about 250 μm to about 50 μm. In certain embodiments, the thickness is from about 150 μm to about 75 μm. In certain embodiments, the thickness of the adhesive layer is from about 125 μm to about 80 μm with 100 μm being preferred. It will be understood that these thickness values are taken prior to application of the article.

A particularly preferred adhesive composition comprises 65% (all percentages noted herein are percentages by weight unless noted otherwise) of a solvent acrylic adhesive and 35% of carboxy methyl cellulose. Another particularly preferred adhesive composition comprises 70% of a solvent acrylic adhesive and 30% of one or more super absorbent polymer materials.

### Release Liner

In one embodiment, the adhesive article includes a release coated liner on the skin-contacting side, which is retained in place prior to use and is removed just prior to application to the user's skin. The release coated liner may be any release coated liner known in the art that is compatible with the pressure sensitive adhesive of the skin-contacting side of the adhesive article. The release liner can be selected from the previously described release liners.

The release liner typically has a thickness of from about 120 μm to about 20 μm, in certain embodiments from about 100 μm to about 70 μm, and in other embodiments from about 70 μm to about 30 μm.

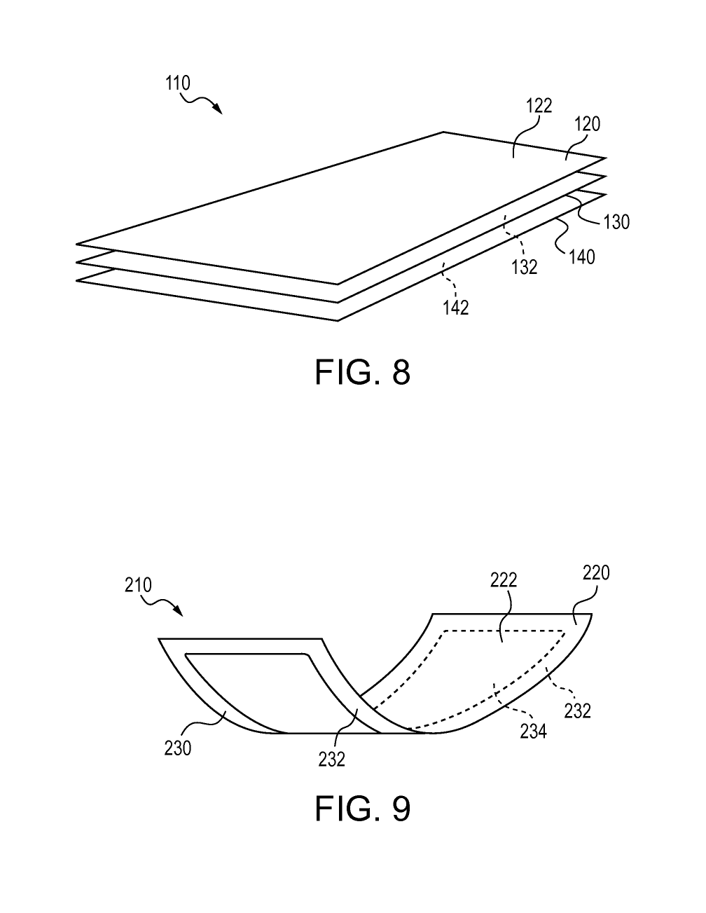

FIG. 8 is an exploded schematic illustration showing a preferred embodiment article 110 comprising a substrate 120, an adhesive layer 130, and a liner 140. The substrate 120 defines an outer face 122. The adhesive 130 is preferably uniformly coated or otherwise applied along the substrate 120 to define an adhesive face 132 oppositely directed from the substrate outer face 122. The liner 140 covers the adhesive face 132. Thus, prior to application of the article 110, the liner 140 is removed to expose the adhesive face 132.

FIG. 9 illustrates another preferred article 210 comprising a substrate 220 and a region of an adhesive layer 230. The substrate 220 defines an outer face 222. The adhesive 230 is preferably coated or applied to the substrate 220 in a pattern fashion so as to define at least one region of adhesive 232 and at least one adhesive-free region 234 along the underside of the article 210. The article 210 is depicted in an arcuate or non-planar configuration in FIG. 9 to depict flexibility of the article. Additional details of adhesive compositions and articles using such are described in US 2010/0322996.

In certain versions, the medical article includes a continuous adhesive layer, i.e., the adhesive layer covers the entirety of the polymeric film or substrate face. In other versions the medical article includes a noncontinuous adhesive layer, such that at least one region of adhesive is disposed on a face of the polymeric film and at least one adhesive-free region is defined on the face. In certain versions using a noncontinuous adhesive layer, an adhesive region in the form of a strip or band is provided that extends around at least a periphery of a face of the polymeric film. One or more adhesive-free regions may be defined on other areas of the polymeric film face such as within a central or interior region of the film.

The various layers and films can be extruded, coated, or otherwise formed by techniques known in the art. Co-extrusion techniques can also be utilized.

The various medial articles described herein are adapted for use in negative pressure wound therapies. And so, the articles include a layer of an adhesive along a face of the polymeric film or substrate which, upon application of the article to a patient, readily forms a seal between the film and the underlying substrate which is typically the patient's skin. It is also preferred that the polymeric film exhibits a relatively high degree of breathability as described herein. Such breathability is indicated by the MVTR values described herein. Furthermore, the articles are sufficiently flexible and conformable so that after application and seal formation, minor dimensional differences along the interface are accommodated by the article so that the seal is maintained. In addition, the adhesive utilized in the articles tends to “swell” after application of the article ad exposure to moisture, water, and/or body fluids. Swelling or increase in overall volume of the adhesive promotes sealing and typically improves the ability of the article to conform to changes in the topography of the underlying surface, e.g., biological skin or tissue. In addition, the articles maintain their adhesive attachment even after contact and exposure to water or other body fluids. Moreover, the articles can be readily configured to receive or interface with one or more conduits or suction lines typically inserted proximate a wound or body area of interest, and under the medical article.

The medical articles are used by removing a release liner or cover layer from the article to expose the adhesive layer. The article is then applied to an area of interest such as an appropriately prepared region along a patient's body. A suction tube or conduit may be placed within the enclosed environment under the article by either forming an aperture or slit in the article and inserting the suction tube therethrough, or by inserting the suction tube between the article and the area of interest. As will be appreciated, the suction tube is in communication with a vacuum pump. Operation of the vacuum pump produces an environment of reduced pressure, i.e., subatmospheric pressure, in the region enclosed by the article.

Additional details concerning negative pressure wound therapy techniques, systems, and equipment are described in one or more of the following patents or published patent applications: US patent publication US 2011/0144599; US 2010/0010477; US 2010/0268198; US 2011/0172612; US 2010/0318043; US 2008/0004549; and U.S. Pat. Nos. 7,534,240; 7,361,184; 7,198,046; 7,909,805; 7,896,823; and 5,645,081.

### Test Methods

Fluid Handling Capacity is a measure of the combined ability of the composite to take up moisture and to evaporate it to the environment. This test is performed by laminating a sample cut to the size of a Paddington cup to the cup on the side having the rubber ring. The circular sealing ring is placed on the sample of the cup and the screws are secured. The cup is weighed (W1). The cup is then turned upside down and filled with 20 ml of a NaCl solution (0.9% wt in deionized water). The metal sealing place is secured to the top side of the cup. The filled cup is weighed (W2). The cup is placed sample side down into an oven at 37° C. for 24 hours. After 24 hours, the cup is removed from the oven and allowed to cool to room temperature for 30 minutes. The cup is then weighed (W3). The metal sealing plate is removed and the cup is emptied. The cup is allowed to stand for 15 minutes on a tissue to remove the NaCl solution, and then weighed (W4). The test conditions are 23° C. (±2°) and 50% (±2%) relative humidity. The Moisture Vapor Transmission Rate (MVTR) equals (W2−W3)×1000. The Static Absorption equal (W4−W1)×1000. The Fluid Handling Capacity (FHC) in g/10 cm²/24 hours is determined as follows:

FHC=(W2−W3)+(W4−W1)

### EXAMPLES

The present subject matter is further described by reference to the following non-limiting examples.

### Examples 1-4

Single Layer Pressure Sensitive Absorbing Adhesives

### Example 1

To 59 parts by weight of a solvent-based acrylic adhesive, Duro-Tak 380-2819 from National Starch at room temperature, is added 40 parts by weight Luquasorb 1010 and 1.0 parts by weight aluminum acetyl acetonate (AAA) crosslinker. The adhesive was coated at a thickness of 120 μm onto a release liner and dried. A polyurethane film backing having a thickness of 25 μm was laminated onto the adhesive layer. Table 1 below shows the Fluid Handling Capacity (FHC), Static absorption and Moisture Vapor Transmission Rate (MVTR) of the adhesive composite.

### Examples 2-4

In a similar manner to Example 1, adhesives are prepared having the compositions shown in Table 1. All amounts are in percent by weight.

TABLE 1
Adhesive Compositions

| | | | | | FHC | Static | MVTR |
|---|---|---|---|---|---|---|---|
| | Acrylic | Luquasorb | | Thickness | (g/m²/ | Absorption | (g/m²/ |
| Example | Adhesive | 1010 | AAA | (μm) | 24 h) | (g/m²/24 h) | 24 h |
| 1 | 59 | 40 | 1 | 120 | 2590 | 1200 | 1390 |
| 2 | 67 | 32 | 1 | 80 | 2135 | 740 | 1395 |
| 3 | 59 | 40 | 1 | 80 | 2130 | 835 | 1295 |
| 4 | 67 | 32 | 1 | 120 | 2100 | 865 | 1235 |

### Examples 5-6

### Multilayer Pressure Sensitive Absorbing Adhesives

### Example 5

A fluid absorbing adhesive made up of 58% by weight DuroTak 380-2819, 40% by weight Luquasorb 1010 and 2% by weight AAA was prepared and coated at a thickness of 150 μm onto a 25 μm thick polyurethane film. A second layer of the adhesive having a thickness of 150 μm was laminated to the first adhesive layer to create an adhesive layer having a total thickness of 300 μm. The composite was sterilized at 25 kGy. Table 2 below shows the FHC, Static absorption and MVTR of the double layer construction (Example 5B) as compared to a construction having a single layer of hydrocolloid adhesive (Example 5A).

TABLE 2
Property Comparisons

| | FHC | Static Absorption | MVTR |
|---|---|---|---|
| Example | (g/m²/24 h) | (g/m²/24 h) | (g/m²/24 h) |
| 5A: 150 μm thick | 2595 | 1315 | 1280 |
| 5B: 300 μm thick | 3760 | 2900 | 860 |

### Example 6

### Example 6A

A multilayer construction was prepared by laminating a 0.3 mm layer of a rubber-based hydrocolloid adhesive onto an 80 μm thick layer of the acrylic hydrocolloid adhesive of Example 5 that had been coated onto a 25 μm thick polyurethane film. The rubber-based hydrocolloid adhesive contained 20% by weight polyisobutylene, 40% by weight sodium carboxy methyl cellulose and 40% by weight rubber phased formed from a 2:8 ratio of physically cross-linked solid rubber (SIS/SI) and a compatible liquid rubber (SI).

### Example 6B

A multilayer construction was prepared substantially in accordance with Example 6A with the exception that the thickness of the acrylic hydrocolloid adhesive layer was 160 μm.

Table 3 below shows the FHC, Static absorption and MVTR of the construction of Example 6A and Example 6B as compared to a construction having only the rubber-based hydrocolloid adhesive (Comparative).

TABLE 3
Property Comparisons

| | FHC | Static Absorption | MVTR |
|---|---|---|---|
| Example | (g/m²/24 h) | (g/m²/24 h) | (g/m²/24 h) |
| Comp. | 3400 | 3050 | 350 |
| 6A | 4445 | 3240 | 1205 |
| 6B | 5125 | 3610 | 1195 |

The results of Table 3 demonstrate that the addition of the thin super absorbent polymer containing adhesive layer, the MVTR is significantly increased, and therefore the fluid handling capacity is much higher without a significant increase in thickness.

### Skin Adhesion

The adhesive composites to be tested were cut into 20 mm×7 cm strips and applied to the inner part of the forearms of each of 6 people. To determine skin adhesion, each strip was removed after a defined wear time at a 90° angle using an Instron adhesion tester at a speed of 300 mm/min. The peel force of each example was measured after 24 hours (Table 4a) of wear time and after 48 hours of wear time (Table 4b).

Example A is a 120 μm thick layer of adhesive containing 67% by weight Duro-Tak 2819 solvent-based acrylic, 32% by weight Luquasorb 1010 and 1% by weight AAA coated onto a backing of 25 μm polyurethane film and sterilized by 25 kGy.

Example B is a 120 μm thick layer of adhesive containing 59% by weight Duro-Tak 2819 solvent-based acrylic, 40% by weight Luquasorb 1010 and 1% by weight AAA coated onto a backing of 25 μm polyurethane film and sterilized by 25 kGy.

Comparative Example C is a commercially available, ultra-thin (140 μm) finger wrap having an MVTR of 502 g/m²/24 h, a static absorption of 900 g/m²/24 h and an FHC of 1402 g/m²/24 h.

TABLE 4a
24 Hour Wear

| | | | | | | | | Std. | | |
|---|---|---|---|---|---|---|---|---|---|---|
| Example | 1 | 2 | 3 | 4 | 5 | 6 | Avg. | Dev. | Max | Min |
| A | 1.94 | 1.77 | 0.88 | 1.78 | 2.21 | 2.74 | 1.89 | 0.48 | 2.74 | 1.78 |
| B | 2.92 | 1.84 | 0.88 | 1.15 | 1.34 | 2.76 | 1.82 | 0.88 | 2.76 | 1.15 |
| C | 0.95 | 0.57 | 0.24 | 0.68 | 1.52 | 0.77 | 0.79 | 0.46 | 1.52 | 0.68 |

TABLE 4b
48 Hour Wear

| | | | | | | | | Std. | | |
|---|---|---|---|---|---|---|---|---|---|---|
| Example | 1 | 2 | 3 | 4 | 5 | 6 | Avg. | Dev. | Max | Min |
| A | 1.33 | 2.37 | lost | 1.39 | 1.76 | 1.89 | 1.75 | 0.42 | 2.37 | 1.33 |
| B | 1.07 | 2.49 | — | 1.79 | 1.27 | 1.54 | 1.43 | 0.65 | 2.49 | 0.79 |
| C | 0.54 | 0.77 | lost | lost | 0.86 | lost | 0.72 | 0.17 | 0.86 | 0.54 |

### Examples 7-12

### Single Layer Pressure Sensitive Absorbing Adhesives

In another set of investigations, a collection of adhesive composite samples were prepared. A polyurethane (PU) film having a thickness of 25 microns was coated with a solvent acrylic adhesive containing either carboxy methyl cellulose or a commercially available polyacrylate. Table 5 set forth below lists the moisture vapor transmission rates (MVTR) for each sample.

TABLE 5
MVTR - Values for Various Preferred
Embodiment Adhesive Composites

| | | | MVTR |
|---|---|---|---|
| Example | PU Film | Adhesive | (g/m²/24 hours) |
| 7 | MEDIFILM 390 | I807/35% A800 | 6100 |
| 8 | INSPIRE 2301 | I807/35% A800 | 4200 |
| 9 | PLATILON U04 | I807/35% A800 | 1500 |
| 10 | MEDIFILM 390 | I807/30% A800 | 4000 |
| 11 | INSPIRE 2301 | I807/30% A800 | 3500 |
| 12 | PLATILON U04 | S2230/40% | 1500 |
| | | LUQUASORB 1010 | |

Referring to Table 5, the polyurethane film was obtained from several commercial sources under the designations MEDIFILM 390 from Mylan Technologies of St. Albans, Vt.; INSPIRE 2301 from Styron LLC of Dow Chemical; and PLATILON U04 from Epurex Films Gmb of Germany. The adhesive contained either carboxy methyl cellulose commercially available under the designation A800 from various suppliers or a polyacrylate commercially available under the designation LUQUASORB 1010 from BASF.

In Table 5, the adhesive composites of Examples 7-8 and 10-11 exhibited MVTR values significantly greater than 2000 g/m²/24 hours.

The fluid absorbing adhesive compositions described herein can be used in a wide array of applications. For example, the adhesives can be used in securement dressings, securement tape and products using such tape, film dressings, ostomy flanges, and adhering sensor patches to a user's skin. In order to obtain long wear times, a composition which provides a relatively high MVTR in conjunction with fluid absorption properties is beneficial. The various compositions described herein will find wide use particularly in medical applications.

Many other benefits will no doubt become apparent from future application and development of this technology.

All patents, published applications, and articles noted herein are hereby incorporated by reference in their entirety.

While the subject matter has been explained in relation to various of its embodiments, it is to be understood that various modifications thereof will be apparent to those skilled in the art upon reading the specification. The features of the various embodiments of the articles described herein may be combined within an article. That is one or more features or aspects of one embodiment may be combined with one or more features or aspects of one or more other embodiments. Therefore, it is to be understood that the subject matter described herein is intended to cover such modifications as fall within the scope of the appended claims.

1. An adhesive composite comprising:
a polymeric backing layer;
a fluid absorbing adhesive layer comprising (a) 20-80% by weight of a solvent-based acrylic pressure sensitive adhesive and (b) 20-80% by weight of at least one gelling agent, wherein the thickness of the adhesive layer is about 40 μm to about 300 μm, and the overall adhesive composite has a moisture vapor transmission rate of at least 2000 g/m²/24 hours.

2. The adhesive composite of claim 1 wherein the gelling agent includes carboxy methyl cellulose.

3. The adhesive composite of claim 1 wherein the gelling agent includes polyacrylate.

4. The adhesive composite of claim 1 wherein the thickness of the adhesive layer is less than about 200 μm.

5. The adhesive composite of claim 1 wherein the thickness of the adhesive layer is from about 80 μm to about 100 μm.

6. The adhesive composite of claim 1 wherein the adhesive layer further comprises a crosslinker.

7. The adhesive composite of claim 1 wherein the gelling agent has an average particle size of less than 70 μm.

8. The adhesive composite of any claim 1 wherein the polymeric backing is breathable.

9. The adhesive composite of claim 1 wherein a static absorption of the composite is greater than about 600 g/m²/24 hours.

10. An adhesive composite comprising:
a polymeric backing layer having a first surface and a second surface;
a first fluid absorbing adhesive layer having a first surface and a second surface comprising (a) a solvent-based acrylic pressure sensitive adhesive and (b) 20-80% by weight of at least one gelling agent having an average particle size of less than 70 μm, wherein the thickness of the adhesive layer is about 40 μm to about 300 μm and the first surface of the first adhesive layer is adhered to the second surface of the backing layer;
a second fluid absorbing adhesive layer having a first surface and a second skin-contacting surface comprising a rubber-based pressure sensitive adhesive, wherein the thickness of the adhesive layer is at least 200 μm and the first surface of the second adhesive layer is adhered to the second surface of the first adhesive layer;
wherein the overall adhesive composite has a fluid handling capacity of at least 4000 g/m²/24 hours.

11. The adhesive composite of claim 10 wherein the thickness of the first adhesive layer is less than about 200 μm.

12. The adhesive composite of claim 10 wherein the first adhesive layer further comprises a crosslinker.

13. The adhesive composite of claim 10 wherein the gelling agent includes carboxy methyl cellulose.

14. The adhesive composite of claim 10 wherein the gelling agent includes polyacrylate.

15. The adhesive composite of claim 10 wherein the polymeric backing is breathable.

16. The adhesive composite according to claim 10 wherein a static absorption of the composite is greater than about 600 g/m²/24 hours.

17. An adhesive composite comprising:
a polymeric backing layer;
a fluid absorbing adhesive layer comprising (a) 20-80% by weight of a solvent-based acrylic pressure sensitive adhesive and (b) 20-80% by weight of at least one gelling agent and wherein the gelling agent has an average particle size of from about 1 μm to about 70 μm.

18. The adhesive composite of claim 17 wherein the thickness of the adhesive layer is about 40 μm to about 300 μm and the composite exhibits a moisture vapor transmission rate of at least 2000 g/m²/24 hours.

19. The adhesive composite of claim 17 wherein the thickness of the adhesive layer is from about 80 μm to about 100 μm.

20. The adhesive composite of claim 17 further comprising a removable release layer on an outer surface of the adhesive layer.

21. The adhesive composite of claim 17 wherein an adhesive control agent is further included in the adhesive layer.

22. The adhesive composite of claim 21 wherein the adhesive control agent is a liquid rubber.

23. The adhesive composite of claim 17 further comprising a pharmaceutically active agent in the adhesive layer.

24. A multilayer medical article adapted for use in negative pressure wound therapy applications, the article comprises:
a polymeric film having a moisture vapor transmission rate (MVTR) of from 1,500 to 14,600 g/m²/24 hours at 38° C.;
a layer of an adhesive composition disposed on the polymeric film, the adhesive composition including (i) at least one adhesive component, and (ii) at least one of a moisture absorbing agent and a hydrocolloid;
wherein the thickness of the adhesive layer is from 50 to 250 μm.

25. The multilayer medical article of claim 24 wherein the adhesive component includes an acrylic adhesive.

26. The multilayer medical article of claim 24 wherein the MVTR of the polymeric film is from 2,500 to 10,000 g/m²/24 hours at 38° C.

27. The multilayer medical article of claim 24 wherein the thickness of the polymeric film is from 10 μm to 75 μm.

28. The multilayer medical article of claim 24 wherein the thickness of the polymeric film is from 15 μm to 45 μm.

29. The multilayer medical article of claim 24 wherein the thickness of the polymeric film is from 20 μm to 30 μm.

30. The multilayer medical article of claim 24 wherein the thickness of the polymeric film is 25 μm.

31. The multilayer medical article of claim 24 wherein the film comprises a material selected from the group consisting of polyurethane, elastomeric polyester, blends of polyurethane and polyester, polyvinyl chloride, polyether-amide block copolymer, porous polyethylene, and combinations thereof.

32. The multilayer medical article of claim 24 wherein the film comprises polyurethane.

33. The multilayer medical article of claim 25 wherein the acrylic adhesive is formed from at least two monomers selected from the group consisting of hydroxyalkyl esters of acrylic or methacrylic acid in which the alkyl group comprises 2 to 4 carbon atoms; alkyl esters of acrylic or methacrylic acid in which the alkyl group of the ester comprises 4 to 18 carbon atoms; α,β-unsaturated monocarboxylic or dicarboxylic acids, their anhydrides and their alkyl or alkenyl esters in which the alkyl group comprises from 1 to 3 carbon atoms and the alkenyl group comprises 2 to 5 carbon atoms; vinyl monomers; monomers containing a functional group selected from amido, amino and epoxy groups; alkylaminoalkyl esters of acrylic or methacrylic acid; and hexamethylene glycol dimethacrylate.

34. The multilayer medical article of claim 24 wherein the adhesive includes a moisture absorbing agent.

35. The multilayer medical article of claim 34 wherein the moisture absorbing agent is a super absorbent polymer.

36. The multilayer medical article of claim 35 wherein the super absorbent polymer absorbs at least 50 times its own weight in water.

37. The multilayer medical article of claim 36 wherein the super absorbent polymer absorbs at least 100 times its own weight in water.

38. The multilayer medical article of claim 37 wherein the super absorbent polymer absorbs at least 150 times its own weight in water.

39. The multilayer medical article of claim 35 wherein the super absorbent polymer exhibits a degree of crosslinking of from 10% to 50%.

40. The multilayer medical article of claim 39 wherein the super absorbent polymer exhibits a degree of crosslinking of from 20% to 40%.

41. The multilayer medical article of claim 24 wherein the moisture absorbing agent is in particulate form and has an average particle size of from 20 μm to 400 μm.

42. The multilayer medical article of claim 41 wherein the moisture absorbing agent has an average particle size of from 20 μm to 200 μm.

43. The multilayer medical article of claim 24 wherein the adhesive includes 20% to 80% by weight of a super absorbing polymer.

44. The multilayer medical article of claim 24 wherein the adhesive includes 40% to 60% by weight of a super absorbing polymer.

45. The multilayer medical article of claim 24 wherein the adhesive includes a hydrocolloid.

46. The multilayer medical article of claim 45 wherein the hydrocolloid is in particulate form and has an average particle size of from 1 μm to 400 μm.

47. The multilayer medical article of claim 46 wherein the average particle size is from 20 μm to 200 μm.

48. The multilayer medical article of claim 24 wherein the thickness of the adhesive layer is from 75 to 150 μm.

49. The multilayer medical article of claim 48 wherein the thickness of the adhesive layer is from 80 to 125 μm.

50. The multilayer medical article of claim 24 further comprising:
a release liner in contact with the adhesive layer.

51. The multilayer medical article of claim 24 wherein the adhesive layer is continuous.

52. The multilayer medical article of claim 24 wherein the adhesive layer is non-continuous and defines at least one adhesive region and at least one adhesive-free region on the polymeric film.

53. The multilayer medical article of claim 52 wherein the adhesive region includes a band of adhesive extending around at least a majority of the perimeter of the polymeric film.

54. The multilayer medical article of claim 53 wherein the adhesive-free region includes a central portion of a face of the polymeric film.

55. A method of producing a multilayer medical article adapted for use in negative pressure wound therapy applications, the method comprising:
providing a polymeric film having a moisture vapor transmission rate (MVTR) of from 1,500 to 14,600 g/m²/24 hours at 38° C.;
providing an adhesive composition including (i) at least one adhesive component and (ii) at least one of a moisture absorbing agent and a hydrocolloid;
forming a layer of the adhesive composition on the film such that the layer has a thickness of from 50 to 250 μm.

56. The method of claim 55 wherein the adhesive component includes an acrylic adhesive.

57. The method of claim 55 wherein forming the layer of the adhesive composition includes forming a continuous layer on the polymeric film.

58. The method of claim 55 wherein forming the layer of the adhesive composition includes forming a noncontinuous layer on the polymeric film.

59. The method of claim 57 wherein the noncontinuous layer includes at least one region of adhesive and at least one adhesive-free region.

60. The method of claim 58 wherein the at least one region of adhesive includes a band of adhesive extending about at least a majority of a perimeter of the polymeric film.

61. A method of forming a sealed region along a biological surface, the method comprising:
providing a multilayer medical article including a polymeric film having a moisture vapor transmission rate (MVTR) of from 1,500 to 14,600 g/m²/24 hours at 38° C., a layer of an adhesive composition disposed on the polymeric film, the adhesive composition disposed on the polymeric film, the adhesive composition including (i) at least one adhesive component, and (ii) at least one of a moisture absorbing agent and a hydrocolloid, wherein the thickness of the adhesive layer is from 50 to 250 μm;
contacting the adhesive layer to the biological surface to thereby form a sealed region between the article and the biological surface.

62. The method of claim 61 wherein the adhesive component includes an acrylic adhesive.
